(19) United States
(12) Patent Application Publication
(10) Pub. No.: US 2010/0179106 A1
(43) Pub. Date: Jul. 15, 2010

(54) MITOCHONDRIAL COMPOSITIONS AND USES THEREOF

(75) Inventor: Shaharyar Khan, Charlottesville, VA (US)

Correspondence Address: Pabst Patent Group LLP, 1545 PEACHTREE STREET NE, SUITE 320, ATLANTA, GA, 30309, US

(73) Assignee: Gencia Corporation, Charlottesville, VA (US)

(21) Appl. No.: 12/676,581

(22) PCT Filed: Sep. 8, 2008
(86) PCT No.: PCT/US2008/075542

§ 371 Date: Mar. 4, 2010

Related U.S. Application Data
(60) Provisional application No. 60/970,683, filed on Sep. 7, 2007.

(51) Int. Cl. A61K 31/66; A61K 47/48; C07F 9/50; A61P 25/00

(52) U.S. Cl. 514/77; 530/300; 536/26.8; 544/258; 544/314; 560/168

(57) ABSTRACT

Compositions and methods for treating mitochondrial disorders are provided. The compositions include compounds having a mitochondrial targeting moiety, for example a lipophilic cation. Certain compounds are effective for increasing the ratio of phosphocreatine/creatine in a host, for example a mammal. Other compounds decrease the ratio of phophocreatine/creating in a host. An exemplary compound is defined by the following structure (Formula Ia): wherein R₁ is H or phosphate and the double bond is between N¹ and C¹ or between N² and C¹; R₂ is a mitochondrial targeting moiety; R₃ an alkyl, alkylaryl, alkylheteroaryl spacer group, a cleavable linker, or absent; R₄ is H or an alkyl, aryl, or heteroaryl group; and R₅ is alkyl, aryl, or heteroaryl; or N¹ C¹, and N together form a heterocyclic ring containing at least 5 atoms, wherein N¹, N³, and R₁-R₅ are as defined above, or N³ and R₅ together form a heterocyclic ring containing at least four atoms; or a pharmaceutically acceptable salt or prodrug thereof.

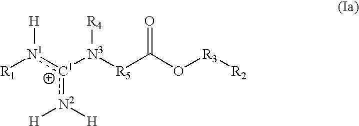

### CROSS REFERENCE TO RELATED APPLICATION

This application claims benefit of and priority to U.S. Provisional Patent Application No. 60/970,683 filed on Sep. 7, 2007, and where permissible is incorporated by reference in its entirety.

### FIELD OF THE INVENTION

Aspects of the invention are generally directed to compositions targeted to the mitochondria and methods of use thereof.

### BACKGROUND OF THE INVENTION

Creatine, or 2-(carbamimidoyl-methyl-amino) acetic acid, is a naturally occurring nitrogenous organic acid that is synthesized in the liver of vertebrates and helps to supply energy to muscle and nerve cells. Creatine is synthesized from the amino acids arginine, methionine and glycine through a two step enzymatic process involving GAMT (guanidinoacetate N-methyltransferase, also known as glycine amidinotransferase) by methylation of guanidoacetate using S-adenosyl-L-methionine (SAM) as the methyl donor. Guanidoacetate itself is formed in the kidneys from the amino acids arginine and glycine. Once made in the liver or acquired through digestion, creatine is stored in cells such as muscle and brain. The mitochondrial enzyme creatine (phospho)kinase, catalyzes the transfer of the phosphate of ATP to creatine, generating creatine phosphate. The reaction is reversible such that when energy demand is high (e.g., during muscle exertion or brain activity) creatine phosphate donates its phosphate to ADP to yield ATP. Both creatine and creatine phosphate are found in muscle, brain and blood.

A normal mixed diet provides approximately 1 to 2 g/day of creatine, with the highest source being meat products (Persky and G A Brazeau, Pharmacol Rev, 53: 161-176 (2001)). The rationale for creatine use in mitochondrial disorders is to increase phosphocreatine (PCr) stores and thus prevent ATP depletion which can occur as a consequence of perturbed mitochondrial function. Patients with mitochondrial myopathies have been shown to have reductions in PCr in muscles and reduced PCr:ATP ratios in the brain (Eleff et al., Proc Natl Acad Sci USA, 81:3529-3533 (1984); Montagna et al., Ann Neurol 31:451-452 (1992); Radda et al., Biochim Biophys Acta, 71:15-19 (1995)). Studies in healthy control subjects and athletes have demonstrated that creatine supplementation is most effective when muscle creatine content is low (Harris et al., Clin Sci (Lond) 83:367-374 (1992)). In a short-term study in patients with mitochondrial disorders, creatine supplementation improved high-intensity anaerobic and aerobic activities, but had no effect on lower-intensity aerobic activities (Tarnopolsky et al., Muscle Nerve 20:1502-1509 (1997)). Although, the long-term beneficial effects of creatine use in mitochondrial disorders is unknown, there have been no reported side effects from dosages as high as 330 g/day (Harris et al., Clin Sci (Lond) 83:367-374 (1992)).

Creatine is a potent agonist of mitochondrial respiration (Walsh et al., Journal of Physiology, 537(Pt 3):971-978 (2001)). Phosphocreatine (PCr) is an important regulator of mitochondrial ADP-stimulated respiration. PCr decreases the sensitivity of mitochondrial respiration to ADP whereas Cr has the opposite effect. During transition from rest to high-intensity exercise, decreases in the PCr/Cr ratio will effectively increase the sensitivity of mitochondrial respiration to ADP. In experiments on brain slice cultures, 20 mM creatine was sufficient to stimulate mitochondrial activity (Li et al., Cell 119:873-887 (2004)). As with manipulating mitochondrial morphology, creatine treatment increases post-synaptic densities as well as activity-dependent morphological plasticity of dendritic spines and neuronal synapses. Thus, pharmacological enhancement of mitochondrial function significantly increases neuronal function and overall metabolic health.

It is an object of the invention to provide compounds targeted to the mitochondria for treating or alleviating at least one symptom of a mitochondrial disorder.

It is another object to provide methods for treating mitochondrial disorders by administering mitochondrial metabolites operably linked to a mitochondrial targeting moiety.

### SUMMARY OF THE INVENTION

Compositions and methods for treating mitochondrial disorders are provided. The compositions include compounds having a mitochondrial targeting moiety, for example a lipophilic cation. Certain compounds are effective for increasing the ratio of phosphocreatine/creatine in a host, for example a mammal. Other compounds decrease the ratio of phophocreatine/creatine in a host. An exemplary compound is defined by the following structure:

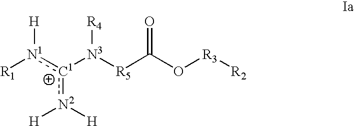

wherein R₁ is H or phosphate and the double bond is between N¹ and C¹ or between N² and C¹;
R₂ is a mitochondrial targeting moiety;
R₃ an alkyl, alkylaryl, alkylheteroaryl spacer group, a cleavable linker, or absent;
R₄ is H or an alkyl, aryl, or heteroaryl group; and
R₅ is alkyl, aryl, or heteroaryl; or

N¹, C¹, and N³ together form a heterocyclic ring containing at least 5 atoms, wherein N¹, N³, and R₁-R₅ are as defined above, or
N³ and R₅ together form a heterocyclic ring containing at least four atoms; or
a pharmaceutically acceptable salt or prodrug thereof.

If the double bond is between C¹ and N², the positive charge typically will reside on N². If the double bond is between C¹ and N¹, the positive charge typically will reside on N¹.

Representative compounds include those of formula Ib and Ic.

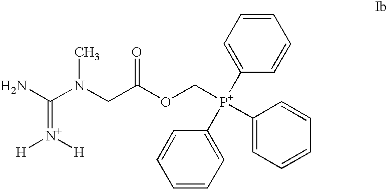

(triphenylphosphonio)methyl N-[amino(iminio)methyl]-N-methylglycinate

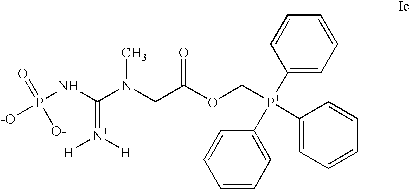

(triphenylphosphonio)methyl N-[amino(iminio)methyl]-N-methylglycinate phosphate

Creatine analogs including a mitochondrial targeting moiety are also provided. Representative creatine analogs that can be modified to include a mitochondrial targeting moiety include, but are not limited to cyclocreatine (1-carboxymethyl-2-iminoimidazolidine), N-phosphorocreatine (N-phosphoryl creatine), cyclocreatine phosphate (3-phosphoryl-1-carboxymethyl-2-iminoimidazolidine), 1-carboxymethyl-2-aminoimidazole, 1-carboxymethyl-2,2-iminomethylimidazolidine, 1-carboxyethyl-2-iminoimidazolidine, N-ethyl-N-amidinoglycine and b-guanidinopropionic acid.

Additional compounds that can be operably linked to a mitochondrial targeting moiety include, but are not limited to folate/folic acid, succinate, orotate, uridine, cytidine, pyruvate, vitamin A/retinoic acid, nicotinamide adenine dinucleotide (NAD+), NADH, nicotinamide adenine dinucleotide phosphate (NADP+), NADPH, ascorbic acid, folate, adenosine, adenosine diphosphate (ADP), adenosine triphosphate (ATP), adenosine monophosphate (AMP), glycerol, nonoate, s-adenosylemthionine (SAM), cyclic guanosine monophosphate (cGMP), cyclic adenosine monophosphate (cAMP), palmitate, acetyl-1-carnitine, alpha-lipoic acid, cardiolipin, cholesterol, acetyl-CoA, acetyl-CoA-SH, malohyl-CoA, glutamate, methylene blue, ascorbate, nitrite, alpha-ketoglutarate, acetate, acetaldehyde, lipoate, glutathione, glyceraldehyde 3-phosphate, malate, oxaloacetate, fumarate, ergocalciferol, cholecalciferol, biotin, valproate/valproic acid, phosphoenol pyruvate, glucose, glucose 6-phosphate, fructose, fructose-6-phosphate, fructose 1,6-bisphosphate, glycogen, UDP-glucose, glucose 1-phosphate, glutamine, glucosamine and analogs.

The compounds described herein may have one or more chiral centers and thus exist as one or more stereoisomers. Such stereoisomers can exist as a single enantiomer, a mixture or enantiomers, a mixture of diastereomers or a racemic mixture.

Compositions containing one or more of the disclosed compounds can be used treat mitochondrial disorders. Mitochondrial myopathies that can be treated include Kearns-Sayre syndrome, Leigh's syndrome, mitochondrial DNA depletion syndrome (MDS), mitochondrial encephalomyopathy, lactic acidosis and strokelike episodes (MELAS), myoclonus epilepsy with ragged red fibers (MERRF), mitochondrial neurogastrointestinal encephalomyopathy (MNGIE), neuropathy, ataxia and retinitis pigmentosa (NARP), and progressive external ophthalmoplegia (PEO).

Additionally, the compounds can be used to treat one or more symptoms of arthritis, congestive heart failure, disuse atrophy, gyrate atrophy, Huntington's disease, and McArdles disease.

### DETAILED DESCRIPTION OF THE INVENTION

### I. Definitions

In describing and claiming the disclosed subject matter, the following terminology will be used in accordance with the definitions set forth below.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C1-C30 for straight chain, C3-C30 for branched chain), and more preferably 20 or fewer. Likewise, preferred cycloalkyls have from 3-10 carbon atoms in their ring structure, and more preferably have 5, 6 or 7 carbons in the ring structure. The alkyl groups can also be substituted with one or more groups including, but not limited to, halogen, hydroxy, amino, thio, ether, ester, carboxy, oxo, and aldehyde groups. The alkyl groups may also contain one or more heteroatoms.

The terms “alkenyl” and “alkynyl” refer specifically to unsaturated aliphatic groups containing one or more double or triple bonds analogous in length (e.g., C₂-C₃₀) and possible substitution to the alkyl groups described above.

“Aryl”, as used herein, refers to 5-, 6- and 7-membered aromatic, heterocyclic, fused aromatic, fused heterocyclic, biaromatic, or biheterocyclic ring system, optionally substituted by halogens, alkyl-, alkenyl-, and alkynyl-groups. Broadly defined, “Ar”, as used herein, includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Alkoxycarbonyl”, as used herein, refers to a substituent having the following chemical formula:

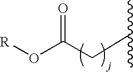

wherein R is a linear, branched, or cyclic alkyl group, wherein j is from about 1 to about 12.

“Alkoxycarbamido”, as used herein, refers to a substituent having the following chemical formula:

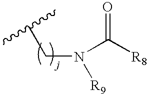

wherein R₈ is alkoxy and R₉ is hydrogen, alkoxy-alkyl, or alkanoyl, and j is from about 1 to about 12.

“Alkylaryl”, as used herein, refers to an alkyl group substituted with an aryl group (e.g., an aromatic or hetero aromatic group).

The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

As used herein, the term “enantiomers” refers to two stereoisomers which are non-superimposable mirror images of one another. As used herein the term “diastereomer” refers to two stereoisomers which are not mirror images but also not superimposable.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-₄)alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Heteroaryl”, as used herein, refers to a monocyclic aromatic ring containing five or six ring atoms consisting of carbon and 1, 2, 3, or 4 heteroatoms each selected from the group consisting of non-peroxide oxygen, sulfur, and N(Y) where Y is absent or is H, O, (C₁-C₈)alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl can be furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

“Halogen”, as used herein, refers to fluorine, chlorine, bromine, or iodine.

The term “host” refers to a multicellular organism having mitochondria including but not limited to mammals such as primates, humans, dogs, cats, cows, pigs, sheep, and the like.

The term “mitochondrial metabolite” refers to an organic compound that is a starting material in, an intermediate in, or an end product of metabolism occurring in the mitochondria.

As used herein, the term “non-nuclear organelle” refers to any cellular membrane bound structure present in a cell, except the nucleus.

As used herein, the term “optical isomer” is equivalent to the term “enantiomer”.

“Operably linked” refers to a juxtaposition wherein the components are configured so as to perform their usual function. For example, a mitochondrial targeting moiety operably linked to compound will direct the linked compound to be localized to the mitochondria. The linked compound maintains biological activity in the mitochondria.

As used herein, the term “organelle” refers to cellular membrane bound structures such as the chloroplast, mitochondrion, and nucleus. The term “organelle” includes natural and synthetic organdies.

The terms ortho, meta and para apply to 1,2-, 1,3- and 1,4-disubstituted benzenes, respectively. For example, the names 1,2-dimethylbenzene and ortho-dimethylbenzene are synonymous.

“Localization Signal or Sequence or Domain” or “Targeting Signal or Sequence or Domain” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location. Exemplary organelle localization signals include nuclear localization signals known in the art and other organelle localization signals known in the art such as those provided in Tables 1 and 2 and described in Emanuelson et al., Predicting Subcellular Localization of Proteins Based on Their N-terminal Amino Acid Sequence. Journal of Molecular Biology. 300(4):1005-16, 2000 Jul. 21, and in Cline and Henry, Import and Routing of Nucleus-encoded Chloroplast Proteins. Annual Review of Cell & Developmental Biology. 12:1-26, 1996, the disclosures of which are incorporated herein by reference in their entirety. It will be appreciated that the entire sequence listed in Tables 1 and 2 need not be included, and modifications including truncations of these sequences are within the scope of the disclosure provided the sequences operate to direct a linked molecule to a specific organelle. Organelle localization signals of the present disclosure can have 80 to 100% homology to the sequences in Tables 1 and 2. One class of suitable organelle localization signals include those that do not interact with the targeted organelle in a receptor:ligand mechanism. For example, organelle localization signals include signals having or conferring a net charge, for example a positive charge. Positively charged signals can be used to target negatively charged organelles such as the mitochondria. Negatively charged signals can be used to target positively charged organdies.

“Pharmaceutically acceptable salt”, as used herein, refer to derivatives of the compounds defined by Formula I, II, and III wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Prodrug”, as used herein, refers to a pharmacological substance (drug) which is administered in an inactive (or significantly less active) form. Once administered, the prodrug is metabolized in the body (in vivo) into the active compound.

The terms “racemate”, “racemic mixture” or “racemic modification” refer to a mixture of equal parts of enantiomers.

As used herein, the term “stereoisomers” refers to compounds made up of the same atoms having the same bond order but having different three-dimensional arrangements of atoms which are not interchangeable. The three-dimensional structures are called configurations.

“Solvate”, as used herein, refers to a compound which is formed by the interaction of molecules of a solute with molecules of a solvent.

As used herein, the term “treating” includes alleviating the symptoms associated with a specific disorder or condition and/or preventing or eliminating said symptoms.

“Cleavable linker”, as used herein, refers to a molecule, moiety, atom, or group of atoms capable of covalently connecting creatine or a creatine analog to a mitochondrial targeting moiety. The term “linker” may refer to a non-peptidyl linker or a peptidyl linker. The linker may optionally have covalently bonded thereto a tether, as defined below, for covalently linking a cytotoxic compound to the linker. The term “peptidyl linker” as used herein refers to a peptide comprising at least two amino acids and which can be coupled to a mitochondrial targeting moiety. The linker may have a reactive group at the carboxyl terminus such as, but not limited to, a chloromethylketone. The peptide of the peptidyl linker may be cleavable by proteolytic enzymes found within a cell. Examples of non-peptidyl linkers include, but are not limited to, disulfide bonds. Non-peptidyl linkers may be cleaved in the presence of absence of an enzyme (e.g., hydrolysis).

### II. Compounds

A. Creatine Compounds Targeted to Mitochondria

Creatine compounds modified to be targeted to the mitochondria are provided. Certain compounds are effective for increasing the ratio of phosphocreatine/creatine in a host, for example a mammal. An exemplary compound is defined by the following structure:

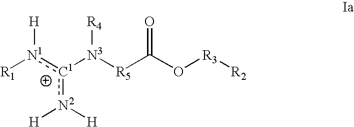

wherein R₁ is H or phosphate and the double bond is between N¹ and C¹ or between N² and C¹;
R₂ is a mitochondrial targeting moiety;
R₃ an alkyl, alkylaryl, alkylheteroaryl spacer group, a cleavable linker, or absent;
R₄ is H or an alkyl, aryl, or heteroaryl group; and
R₅ is alkyl, aryl, or heteroaryl; or

N¹, C¹, and N³ together form a heterocyclic ring containing at least 5 atoms, wherein N¹, N³, and R₁-R₅ are as defined above, or
N³ and R₅ together form a heterocyclic ring containing at least four atoms; or
a pharmaceutically acceptable salt or prodrug thereof.

Representative compounds include those of formula Ib and Ic.

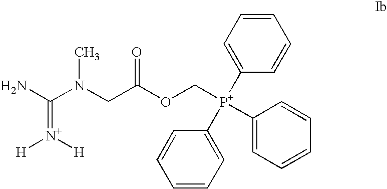

(triphenylphosphonio)methyl N-[amino(iminio)methyl]-N-methylglycinate

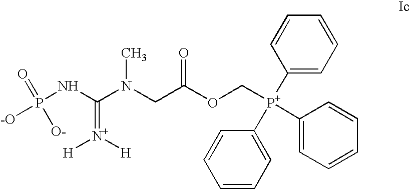

(triphenylphosphonio)methyl N-[amino(iminio)methyl]-N-methylglycinate phosphate.

Suitable compounds containing a mitochondrial targeting moiety can also be prepared from creatine analogs including, but not limited to, the analogs shown in the table below. Generally, the mitochondrial targeting moiety is covalently coupled to creatine or a creatine analog via the carboxylic acid group.

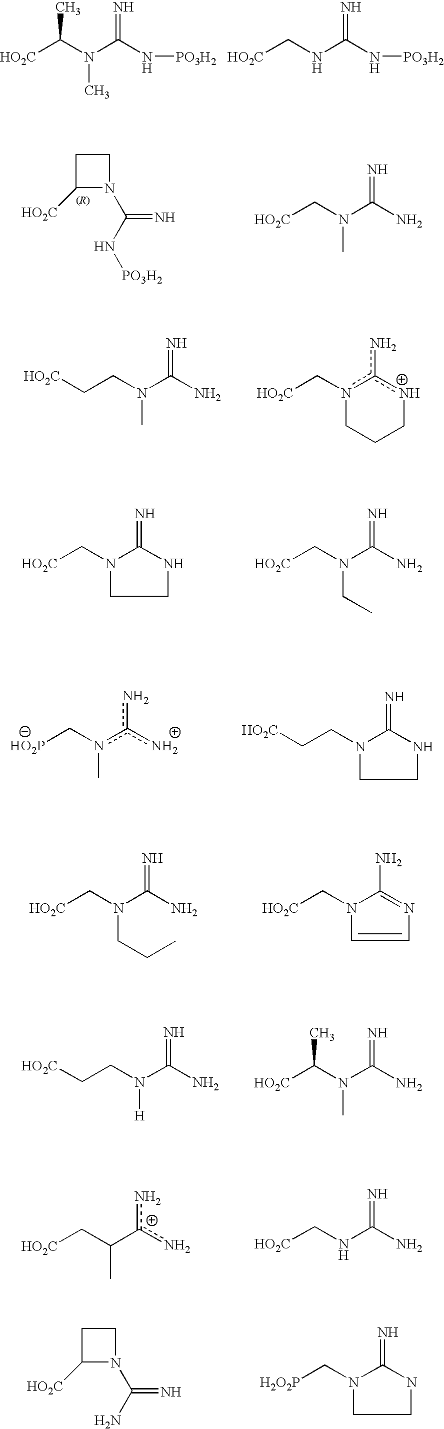

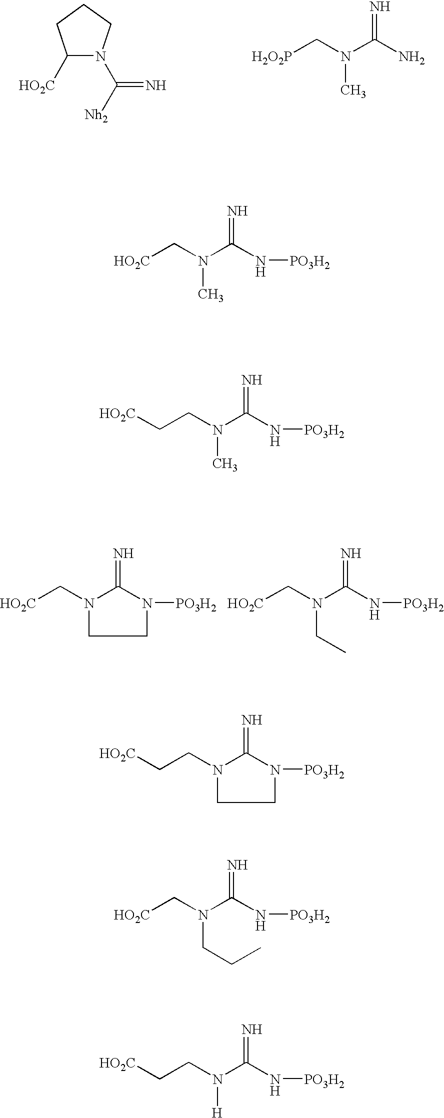

Other exemplary creatine analogs that can be modified to include a mitochondrial targeting moiety include, but are not limited to cyclocreatine (1-carboxymethyl-2-iminoimidazolidine), N-phosphorocreatine (N-phosphoryl creatine), cyclocreatine phosphate (3-phosphoryl-1-carboxymethyl-2-iminoimidazolidine), 1-carboxymethyl-2-aminoimidazole, 1-carboxymethyl-2,2-iminomethylimidazolidine, 1-carboxyethyl-2-iminoimidazolidine, N-ethyl-N-amidinoglycine and b-guanidinopropionic acid.

The compounds described herein may have one or more chiral centers and thus exist as one or more stereoisomers. Such stereoisomers can exist as a single enantiomer, a mixture of diastereomers or a racemic mixture.

B. Additional Compounds

Additional compounds that can be targeted to the mitochondria include, but are not limited to folate/folic acid, succinate, orotate, uridine, cytidine, pyruvate, vitamin A/retinoic acid, and valproate/valproic acid. Compounds having a carboxylic acid can be operably linked to a mitochondrial targeting moiety, for example lipophilic cations discussed below. These compounds can be used to modulate mitochondrial activity. Valproic acid derivatives can be used to downregulate or inhibit mitochondrial activity. Downregulation or inhibition of mitochondrial activity can be useful in the treatment of epilepsy and schizophrenia. Compounds without a carboxylic acid can be chemically modified to have a carboxylic acid which can then be linked to a lipophilic cation.

C. Mitochondrial Targeting Moiety

The disclosed compositions include one or more mitochondrial targeting moieties. Mitochondrial targeting moieties are known in the art and include lipophilic cations as well as small molecules that convey a positive charge to the compound under physiological conditions. Representative mitochondrial targeting moieties include, but are not limited to alkyltriphenylphosphonium, tetraphenylphosphonium, tetraphenylarsonium, tribenzyl ammonium, phosphonium, polyarginine, polylysine, and delocalized lipophilic cations containing one to three carbimino, sulfimino, or phosphinimino units as described in Kolomeitsev et al., Tet. Let., Vol. 44, No. 33, 5795-5798 (2003). Suitable alkyltriphenylphosphonium moieties include, but are not limited to, those alkyltriphenylphosphonium moieties containing a C₁-C₆ straight chain alkylene group having from 1 to 6 carbons, such as a methylene, ethylene, propylene, or butylenes group.

Liphophilic cations are preferred mitochondrial targeting moieties because they can pass directly through phospholipid bilayers without requiring a specific uptake mechanism, and they accumulate substantially within mitochondria due to the large membrane potential. The large hydrophobic radius of the TPP cation enables it to pass easily through the phospholipid bilayer relative to other cations. In one embodiment the disclosed compounds include TPP derivatives modified to increase hydrophobicity. For example, the hydrophobicity of the targeting moiety can be increased by increasing the length of the carbon chain linker as described in Asin-Cayuela et al., FEBS Lett., 30:571 (1-3), 9-16 (2004).

Without wishing to be bound to one theory, it is believed that lipophilic cations are taken up from a positively charged cellular compartment into a negatively charged compartment until a sufficiently large concentration gradient is built up to equalize the electrochemical potential of the molecules in the two compartments. For every 60 mV increase in membrane potential, there will be approximately tenfold accumulation of the lipophilic cation within mitochondria. Because the plasma membrane has a negative 30-60 mV potential on the inside, lipophilic cations will accumulate 5 to 10 fold in the cytosol. Lipophilic cations within the cytosol will accumulate in mitochondria because the mitochondrial membrane potential is typically about 140 to 180 mV.

D. Linking Group

The mitochondrial targeting moiety can be linked directly to the disclosed compounds or through a spacer group. Spacer groups include alkyl alkylaryl or alkylheteroaryl spacer groups having a chain length from about C₁ to about C₁₂, or a subrange thereof such as C₁ to C₃, C₂ to C₄ etc. The spacer group is optionally substituted by one or more double and/or triple bonds. In certain aspects the total number of atoms in the alkylaryl and alkylheteroaryl groups is from about 6 to about 50.

The spacer group can optionally include a cleavable linkage or bond so that the mitochondrial targeting moiety can be cleaved once the compound enters the mitochondrion. Representative cleavable linkages include, but are not limited to, amide bonds, ester bonds, and disulfide bonds.

### III. Methods of Manufacture

Synthesis of alkyltriphenylphosphonium cations are known in the art. See for example M P Murphy and R A Smith. Annu Rev Pharmacol Toxicol. 2007; 47:629-56 (2007). The compounds described herein can be synthesized by reacting creatine or a creatine analog with a lipophilic cation. For example, the carboxylic acid group in creatine or a creatine analog can be converted to a more electrophilic group, such as an acid chloride or ester. The modified creatine or creatine analog can then be reacted with triphenyl phosphonine to form the compounds described herein. Alternatively, the carboxylic acid group in creatine or a creatine analog, or a more reactive functional group can be reacted with a grignard reagent containing an electrophilic group to introducing an alkyl or aryl spacer. This compound can then be reacted with triphenyl phosphine to form the compounds described herein.

In yet another embodiment, haloalkyltriphenylphosphonium salt such as Ph₃P+(CH₂)₄I reacts with a deprotonated hydroxyl group on a creatine compound to form the TPP cation conjugated via a 4-carbon alkyl chain and an ether or ester bond.

Exemplary reaction schemes for the synthesis of the compounds described herein are show below:

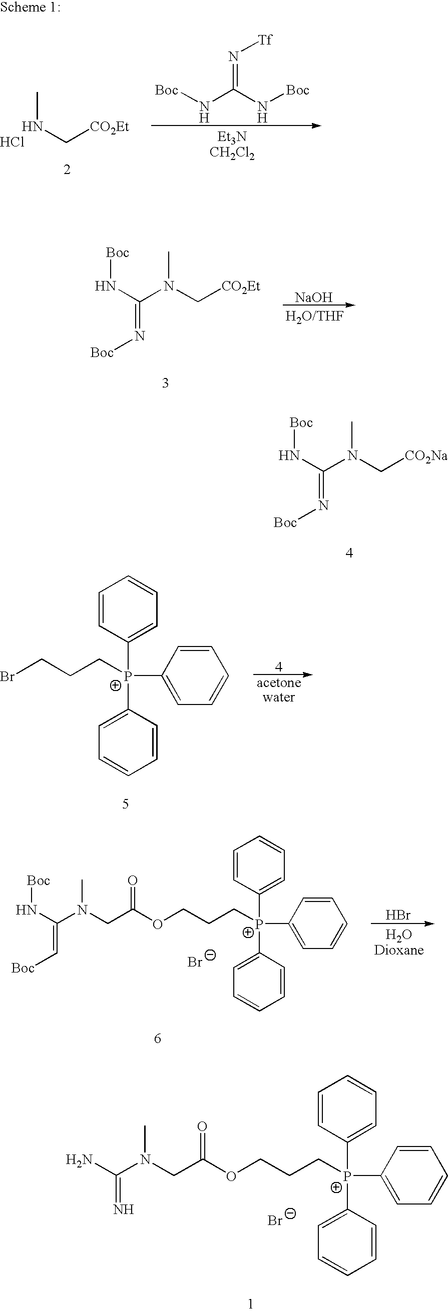

Reaction of commercially available sarcosine ester hydrochloride (1) with N,N′-di-Boc-N″-trifylguanidine should afford the protected guanidine (3) (Baker et al., Org. Syn., 78, 91098 (2002)). Ester hydrolysis of the protected guanidine with aqueous sodium hydroxide should produce the sodium carboxylate (4). Reaction of (4) with commercially available 3-bromopropyl triphenylphosphonium bromide (5) should afford the corresponding ester (6) (Schweizer and Creasy, J. Org. Chem., 36, 2379-2381 (1971)). Deprotection of the ester under acidic conditions should give the phosphonium salt (1).

Alternatively, the phosphonium salt (1) may be prepared by the syntheses described in Scheme 2 or Scheme 3.

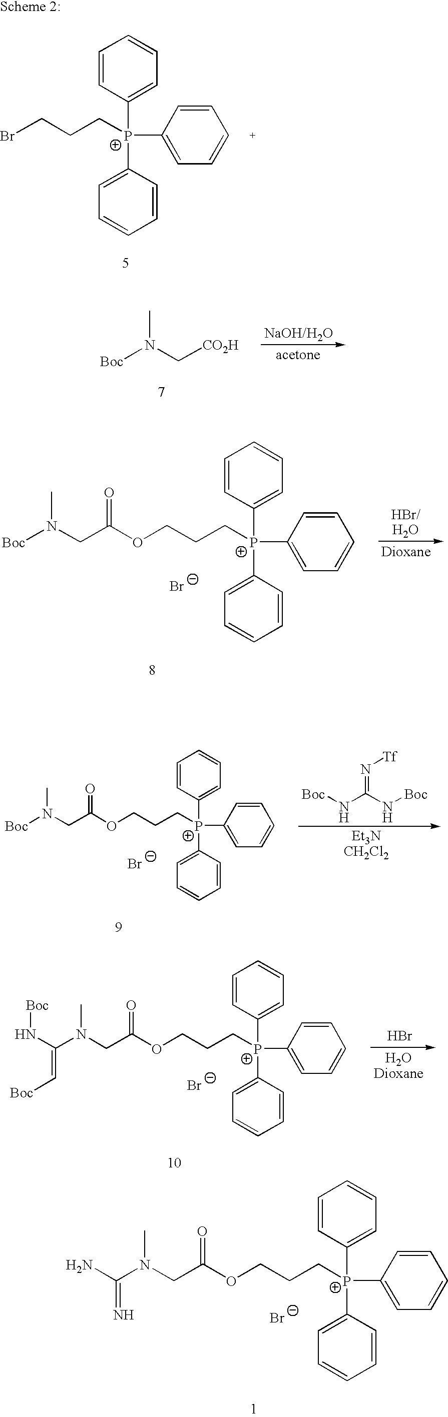

Commercially available 3-bromopropyl triphenylphosphonium bromide (5) is reacted with commercially available Boc-sarcosine (7) in the presence of a stoichiometric sodium hydroxide to afford the corresponding ester (8). Removal of the Boc group using dilute HBr should afford the phosphonium salt (9). Reaction of the phosphonium salt (9) with the protected guanidine triflamide should afford guanidine (10). Final deprotection should produce the phosphonium salt (1).

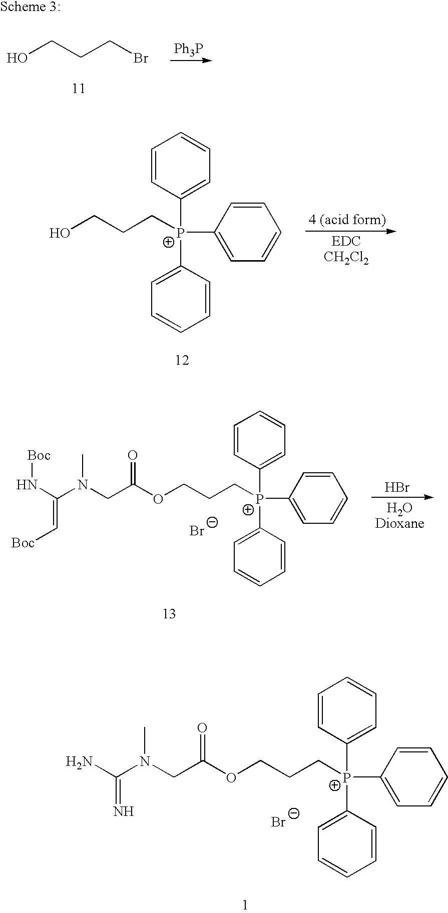

3-hydroxypropyl triphenylphosphonium bromide (12) is prepared from 3-bromopropanol and triphenylphosphine use a procedure well known in the art (Page et al., Synlett., 1022-1024 (2003); Ceruti et al., J. Med. Chem., 35, 3050-3058 (1992)). The alcohol (12) will be coupled to sarcosine to form the corresponding ester (13). Deprotection of the ester (12) should give phosphonium salt (1).

### IV. Formulations

Formulations containing one or more of the compounds described herein may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

A. Other Active Agents

The compositions optionally contain one or more additional active agents. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones.

1. Antibiotics

Representative antibiotics include, without limitation, benzoyl peroxide, octopirox, erythromycin, zinc, tetracyclin, triclosan, azelaic acid and its derivatives, phenoxy ethanol and phenoxy proponol, ethylacetate, clindamycin and meclocycline; sebostats such as flavinoids; alpha and beta hydroxy acids; and bile salts such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin.

In one embodiment, the concentration of the antibiotic is from about 0.01% to about 20%, preferably from about 1% to about 15%, more preferably from about 6% to about 12% by weight of the final composition.

2. Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical application.

In one embodiment, the concentration of the non-steroidal anti-inflammatory agent is from about 0.01% to about 20%, preferably from about 1% to about 15%, more preferably from about 6% to about 12% by weight of the final composition.

3. Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

In one embodiment, the concentration of the steroidal anti-inflammatory agent is from about 0.01% to about 20%, preferably from about 1% to about 15%, more preferably from about 6% to about 12% by weight of the final composition.

4. Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

In one embodiment, the concentration of the anti-microbial agent is from about 0.01% to about 20%, preferably from about 1% to about 15%, more preferably from about 6% to about 12% by weight of the final composition.

B. Dosages

For all of the creatine compounds disclosed, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 10 mg/kg of body weight daily are administered to mammals. Generally, for intravenous injection or infusion, dosage may be lower.

### V. Methods of Treatment

A. Mitochondrial Myopathies

Embodiments of the present disclosure provide compositions and methods for targeted delivery of compounds to mitochondria to modulate mitochondrial function or treat one or more symptoms of a mitochondrial disorder. Suitable mitochondrial disorders that can be treated with the compositions disclosed herein include but are not limited to mitochondrial myopathies. Mitochondrial myopathies include Kearns-Sayre syndrome, Leigh's syndrome, mitochondrial DNA depletion syndrome (MDS), mitochondrial encephalomyopathy, lactic acidosis and strokelike episodes (MELAS), myoclonus epilepsy with ragged red fibers (MERRF), mitochondrial neurogastrointestinal encephalomyopathy (MNGIE), neuropathy, ataxia and retinitis pigmentosa (NARP), and progressive external ophthalmoplegia (PEO).

The disclosed compositions can be used to modulate ATP production in mitochondria by altering the ratio of phosphocreatine/creatine. The ratio of phosphocreatine/creatine can be increased relative to a control by administering the one or more of the disclosed compounds. Increasing the amount of phosphocreatine in the mitochondria increases the ability of the mitochondria to produce ATP. Thus, another embodiment provides a method for increasing mitochondrial production of ATP in a host by administering to the host an effective amount of the disclosed compositions. Increasing the ATP-generating capacity allows a cell to better handle energetic challenges, thus preventing cell damage or death, improving cellular function, increasing cellular healing and replacement, and preventing tumorigenesis.

B. Additional Disorders

The disclosed composition can also be used to treat one or more symptoms associated with arthritis, congestive heart failure, disuse atrophy, gyrate atrophy, Huntington's disease, McArdles disease, Alexander disease, Alzheimers, Parkinsons, Amino Acid disorders, Ataxias, Barth, Tafazzins, Cardiomyopathy, Carnitine disorders, Cartilage-Hair hypoplasia, Congenital muscular dystrophy, cramps, HAM, MELAS, MERRF, Non-syndromic and amino-glycoside induced deafness, DIDMOAD, Deafness-Dystonia, Diabetes, Dystonia, Encephalopathies, Blindness, macular degeneration, LHON, Gyrate atrophy, Optic atrophy, Wolfram, External Ophthalmoplegia, HyperThyroid, Fatigue, Exercise intolerance, Friedreich ataxia, Huntington's, Hypoglycemia, Kearns-Sayre, Leigh's syndrome, Leukodystrophy, Maple syrup urine disease, Menkes, MILS, MNGIE, Multiple symmetric lipomatosis, Myalgias, Myoglobinuria, Inclusion body myositis, NARP/MILS, Neoplasms, Sensory neuropathy, Occipital horn syndrome, Paraganglioma, Pearson's, Rhabdomyolysis, Spastic paraparesis, Spinal muscular atrophy, Stuve-Wiedemann syndrome, Sudden infant death (SIDS), Wilson's disease, cancer, COPD, stroke, cardiac infarction, and inflammation.

The disclosed compositions can also be used for iatrogenic indications—HAART therapies, amino-glycoside antibiotics, COX-2 inhibitor related cardiac disease.

One embodiment provides a nutraceutical including one or more of the disclosed mitochondria-targeted compounds. The nutraceucitcal can be used for endurance training, muscle/strength building, bone density increase, cognitive function, wound healing, anti-aging, anti-obesity/weight loss, anti-ROS.

C. Administration

Pharmaceutical compositions including the disclosed compounds are provided. The pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration. In a preferred embodiment, the compounds are administered orally. In another embodiment, the compounds are administered parenterally in an aqueous solution. In general, pharmaceutical compositions are provided including effective amounts of a creatine compounds or analogs.

1. Oral Delivery

The compositions can be formulated for oral delivery. Oral solid dosage forms are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets, pellets, powders, or granules. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the ABC transporter ligands (or chemically modified forms thereof) and inert ingredients which allow for protection against the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

The compositions may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the peptide (or derivative) or by release of the peptide (or derivative) beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used.

The active ingredient (or derivative) can be included in the formulation as fine multiparticulates in the form of granules or pellets of particle size about 1 mm. The formulation of the material for capsule administration could also be as a powder, lightly compressed plugs, or even as tablets. These therapeutics could be prepared by compression.

Colorants and/or flavoring agents may also be included. For example, the composition may be formulated (such as by liposome or microsphere encapsulation) and then further contained within an edible product, such as a refrigerated beverage containing colorants and flavoring agents.

2. Parenteral Delivery

Preparations disclosed here for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

3. Mucous Membrane Delivery

Compositions for rectal or vaginal administration are preferably suppositories which may contain, in addition to the active substance, excipients such as cocoa butter or a suppository wax. Compositions for nasal or sublingual administration are also prepared with standard excipients well known in the art.

Those skilled in the art will recognize, or be able to ascertain using no more than routine experimentation, many equivalents to the specific embodiments of the invention described herein. Such equivalents are intended to be encompassed by the following claims.

I claim:

1. A compound having the following structure: 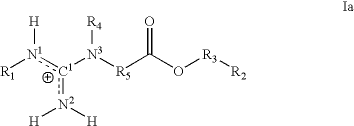
wherein R₁ is H or phosphate and the double bond is between N¹ and C¹ or between N² and C¹;
R₂ is a mitochondrial targeting moiety;
R₃ an alkyl, alkylaryl, alkylheteroaryl spacer group, a cleavable linker, or absent;
R₄ is H or an alkyl, aryl, or heteroaryl group; and
R₅ is alkyl, aryl, or heteroaryl; or
N¹, C¹, and N³ together form a heterocyclic ring containing at least 5 atoms, wherein N¹, N³, and R₁-R₅ are as defined above, or
N³ and R₅ together form a heterocyclic ring containing at least four atoms; or
a pharmaceutically acceptable salt or prodrug thereof.

2. The compound of claim 1 wherein the mitochondrial targeting moiety comprises a lipophilic cation.

3. The compound of claim 2 wherein the lipophilic cation comprises triphenylphosphonium.

4. The compound of claim 1 wherein the mitochondrial targeting moiety comprises polyarginine or polylysine.

5. The compound of claim 1 wherein the spacer group comprises a cleavable linkage.

6. The compound of claim 3 wherein the cleavable linkage is an ester linkage.

7. The compound of claim 1 having the following structure:

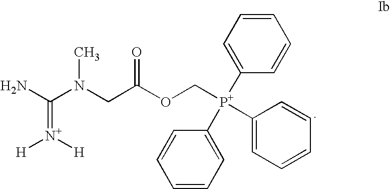

8. The compound of claim 1 having the following formula:

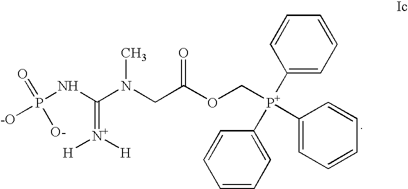

9. The compound of claim 1, wherein the compound is (triphenylphosphonio)methyl N-[amino(iminio)methyl]-N-methylglycinate or a pharmaceutically acceptable salt or prodrug thereof.

10. A pharmaceutical composition comprising the compound of anyone of claims 1-9 and a pharmaceutically acceptable excipient.

11. A method for increasing levels of phosphocreatine in a host comprising administering to the host the compound of any one of claims 1-9 or the pharmaceutical composition of claim 10.

12. A method for treating mitochondrial myopathy comprising
administering to a host having or suspected of having a mitochondrial myopathy the compound of any one of claims 1-10.

13. The method of claim 12 wherein the compound is administered in an amount effective to increase phosphocreatine levels in the host relative to a control.

14. The method of claim 13 wherein the increase in phosphocreatine levels occurs in mitochondria of the host.

15. The method of claim 12 wherein the mitochondrial myopathy is selected from the group consisting of Kearns-Sayre syndrome, Leigh's syndrome, mitochondrial DNA depletion syndrome (MDS), mitochondrial encephalomyopathy, lactic acidosis and strokelike episodes (MELAS), myoclonus epilepsy with ragged red fibers (MERRF), mitochondrial neurogastrointestinal encephalomyopathy (MNGIE), neuropathy, ataxia and retinitis pigmentosa (NARP), and progressive external ophthalmoplegia (PEO).

16. A mitochondrial metabolite operably linked to a mitochondrial targeting moiety.

17. The mitochondrial metabolite of claim 16 wherein the mitochondrial targeting moiety is a lipophilic cation.

18. The mitochondrial metabolite of claim 17 wherein the lipophilic cation is a triphenylphosphonium ion.

19. The mitochondrial metabolite of claim 16 wherein the mitochondrial metabolite is selected from the group consisting of folate/folic acid, succinate, orotate, uridine, cytidine, pyruvate, vitamin A/retinoic acid, nicotinamide adenine dinucleotide (NAD+), NADH, nicotinamide adenine dinucleotide phosphate (NADP+), NADPH, ascorbic acid, folate, adenosine, adenosine diphosphate (ADP), adenosine triphosphate (ATP), adenosine monophosphate (AMP), glycerol, nonoate, s-adenosylemthionine (SAM), cyclic guano sine monophosphate (cGMP), cyclic adenosine monophosphate (cAMP), palmitate, acetyl-1-carnitine, alpha-lipoic acid, cardiolipin, cholesterol, acetyl-CoA, acetyl-CoA-SH, malohyl-CoA, glutamate, methylene blue, ascorbate, nitrite, alpha-ketoglutarate, acetate, acetaldehyde, lipoate, glutathione, glyceraldehyde 3-phosphate, malate, oxaloacetate, fumarate, ergocalciferol, cholecalciferol, biotin, valproate/valproic acid, phosphoenol pyruvate, glucose, glucose 6-phosphate, fructose, fructose-6-phosphate, fructose 1,6-bisphosphate, glycogen, UDP-glucose, glucose 1-phosphate, glutamine, glucosamine and analogs.

20. A method of treating a mitochondrial disorder comprising administering to a host an effective amount of the mitochondrial metabolite according to any one of claims 16-19 to alleviate one or more symptoms of the mitochondrial disorder.
